(19) United States
(12) Patent Application Publication
(10) Pub. No.: US 2026/0098240 A1
(43) Pub. Date: Apr. 9, 2026

(54) Automated microbiological culture dish processing machine and processing method

(71) Applicant: ALLIANCE BIO EXPERTISE, BRUZ (FR)

(72) Inventors: Arnaud FOURMOND, Rennes (FR); Johan DEVELON, LE Rheu (FR); Stéphane BONILLA, Rennes (FR)

(21) Appl. No.: 19/348,909

(22) Filed: Oct. 3, 2025

(30) Foreign Application Priority Data
Oct. 3, 2024 (FR) FR2410664
Oct. 1, 2025 (FR) FR2511270

(51) Int. Cl. C12M 1/36; C12M 1/00; C12M 1/22; C12M 3/00

(52) U.S. Cl. CPC C12M 41/48; C12M 23/10; C12M 23/38; C12M 23/40; C12M 23/48

(57) ABSTRACT

An automated culture dish processing machine, which includes an operational unit designed to receive culture dishes in continuous succession and to carry out a series of successive treatments on each of the culture dishes. The operational unit includes a plurality of stationary processing stations arranged at predetermined intervals around an axis and capable of being activated at least partially simultaneously, and a drive device for rotating the culture dishes relative to the processing stations, provided with housings arranged corresponding to the intervals of the processing stations, such that a rotation of the drive device about the axis successively positions a given culture dish at each of the processing stations.

### CROSS REFERENCE TO RELATED APPLICATIONS

The present application claims priority to French Patent Application No. FR2410664, filed Oct. 3, 2024, and FR2511270, filed Oct. 1, 2025, the contents of which are incorporated herein by reference in their entireties.

### TECHNICAL FIELD

The invention falls within the field of instrumentation intended for biology and microbiology laboratories. More specifically, the invention relates to an automation solution for laboratory equipment suitable for reading and sorting biological or microbiological culture dishes.

The invention has numerous applications in particular, but not exclusively, in the pharmaceutical, cosmetic, agro-food, veterinary and clinical sectors.

### TECHNOLOGICAL BACKGROUND

More specifically, the remainder of this document focuses on describing the problem that exists in the field of food microbiological analysis. Of course, the invention is not limited to this specific application context, but is relevant to any laboratory automation solution that encounters a similar issue.

The demand for the automation of laboratory practices has increased significantly in recent years, especially in the agri-food sector where process safety, productivity and reliability have become paramount.

To ensure consumer safety, food and its environment are regularly subjected to microbiological checks at various stages of production. The objective of these checks is to obtain as efficiently and safely as possible information on the presence or absence of pathogenic micro-organisms in the food samples taken and, where appropriate, information on the quantity of these pathogenic micro-organisms, commonly referred to as “colonies”.

Laboratories have to process a substantial amount of samples daily, typically between 1,000 and 10,000 Petri dishes per day depending on their size and needs. However, the various steps of this processing are still mostly carried out manually by a qualified laboratory technician. For the search for a given pathogenic microorganism, such as Listeria monocytogenes (a bacteria responsible for listeriosis in humans), the technician first scans the bar code affixed to the Petri dish using a bar code reader. The technician then carries out a microbiological analysis of the sample to look for and identify any pathogenic microorganisms. They then manually enter the results on a computer. Colony counting can also be performed visually by the technician. Finally, the latter performs a manual sorting of the Petri dishes identified as positive (i.e. with the presence of microorganisms) or negative (i.e. with the absence of microorganisms).

These operations are repetitive and time-consuming, which can lead to handling errors or cross-contamination between samples, or even sample loss, which is not optimal. Furthermore, one of the requirements of laboratories is to free up time for skilled technicians so that it can be redirected towards more value-added and less tedious tasks.

Petri dish reading devices have been on the market for about fifteen years. The majority of these devices are specifically designed for a relatively limited type of microorganisms, based on fixed and homogeneous conditions. A major limitation of these devices is their inability to perform all the tasks necessary for the dish reading process. In fact, the scanning rate is relatively low (typically around 8 to 10 seconds) because the technician has to add the steps necessary to identify the sample (manual reading of the bar code, and entering the reading results).

Due to the diversity of culture media, samples and colony forms, various parameters have to be adjusted to adapt the analysis and the output of results. On a daily basis, even with pre-configured setting models, it is necessary to adjust certain parameters to enable accurate reading and/or counting of colonies present in the Petri dishes. The technician must therefore adjust the parameters empirically.

Furthermore, there are automatic Petri dish processing devices in the prior art that combine artificial intelligence for the identification and counting of colonies with a robotic arm for loading and unloading Petri dishes, working in conjunction with the Petri dish reader. Such devices produce processing rates of between 100 and 150 dishes per hour; however, these figures still fall short of the rate targets set by microbiological analysis laboratories, particularly in the food industry, where the desired processing rates are typically between 700 and 900 dishes per hour (unless the number of devices in the laboratories is increased, which is not feasible due to cost and space constraints). In addition, the use of a robotic arm is relatively complex and costly to implement.

Finally, new generations of incubators have recently been introduced on the market, featuring the integration of ultra-responsive high-end video image sensors designed to provide near real-time information on the presence of microbial colonies in Petri dishes. However, the processing capacity of such incubators is still limited to 500 dishes per day, with a high implementation cost.

There is therefore a genuine need to provide an efficient, robust, and versatile automation solution for reading and sorting culture dishes, particularly one that achieves a higher level of productivity than currently available, while ensuring accurate and reproducible results.

### DISCLOSURE OF THE INVENTION

The present invention proposes a solution for overcoming disadvantages of the prior art.

In one specific embodiment of the invention, an automated machine for processing culture dishes is proposed, characterised in that it comprises:
a first storage carousel mounted for rotation about a first axis, designed for the storage of culture dishes to be processed;
a second storage carousel mounted fixed about a second axis;
an operational unit designed to receive culture dishes in continuous succession from the first carousel and to carry out a series of successive treatments on each of the received culture dishes, said operational unit comprising:
   a plurality of stationary processing stations arranged at predetermined intervals around the second axis, with at least some of said processing stations being capable of being activated at least partially simultaneously, said processing stations comprising:
      a receiving station intended to receive a culture dish to be processed from the first carousel;
      an analysis and allocation station designed to analyse the contents of the received culture dish and allocate it a specific type from at least two predefined dish types based on the analysis results;
      at least one first and one second transfer station designed to transfer the culture dish to either the first or second storage carousel, depending on the type allocated to said culture dish;
   a drive device for rotating the culture dishes relative to the processing stations, said drive device comprising housings for the culture dishes arranged at intervals corresponding to those of the processing stations, such that a rotation of the drive device about the second axis successively positions a given culture dish at each of the processing stations.

Thus, the invention is based on a comprehensive and efficient automation solution for all tasks involved in the analysis and sorting of culture dishes. This approach involves equipping the processing machine with an operational unit with multiple processing stations, intended to receive the culture dishes in continuous succession and to automatically carry out a cycle of successive treatments on each of the dishes, all or some of the processing stations being able to be activated at least partially simultaneously. Each processing station has a specific role in the processing cycle as the culture dishes are brought to each of the stations, allowing for parallel processing of the dishes and ultimately optimising the processing rate, while ensuring accurate and reproducible analysis results.

Indeed, the duration of the treatments carried out is not always the same; some treatments can be performed concurrently, but naturally, if a treatment has a shorter duration, it will finish before a longer treatment or start after the beginning of the long processing station. In other words, two stations with identical processing times will be activated simultaneously, whereas for two stations with different processing times, the short processing station will be activated partially concurrently with the long processing station. Thus, depending on a specific embodiment, the treatment duration between two rotations of the culture dishes is determined by the station that requires the longest treatment duration.

According to one specific embodiment, each transfer station is assigned to a distinct type of culture dish (for example, a first station is assigned to positive dishes and a second station to negative dishes). According to an alternative embodiment, at least some transfer stations are assigned to the same type of culture dish (for example, two first stations are assigned to “positive” dishes, while a second station is assigned to “negative” dishes), which allows for an increase in storage capacity for a given type of culture dish.

According to one specific feature, said drive device comprises an upper drive plate and a lower drive plate arranged parallel to one another and integral with one another, the upper and lower drive plates being mounted for rotation about the second axis and comprising a set of upper and lower Petri dish housings respectively, the upper and lower housings being evenly distributed on the periphery of the upper and lower drive plates around the second axis, and arranged facing each other in pairs.

Thus, the machine features a double drive plate to transport the culture dishes from one processing station to another, with the option of positioning them on two distinct levels, while ensuring that the base of each dish remains paired with its lid.

According to one specific feature, said drive device also comprises an upper sliding surface fixedly mounted in relation to the second axis and extending partially along the lower face of the upper drive plate, and a lower sliding surface fixedly mounted in relation to the second axis and extending along the lower face of the lower drive plate.

According to a first embodiment of the drive device, the culture dishes to be processed each being provided with a lid attached to a base and positioned in a so-called upright position in the first carousel:
the upper housings are each provided with a through-hole dimensioned to allow the passage of a culture dish and the lower end of which ends with an annular shoulder dimensioned to retain the lid in the upper housing and allow the base to separate from the lid due to gravity in the absence of the upper sliding surface, the upper sliding surface extending partially under the upper drive plate to hold the base attached to the lid on at least one of the processing stations;
the lower housings are each provided with a through-hole dimensioned to allow the passage of a culture dish base, the lower sliding surface extending under the lower drive plate to secure the base in the lower housing.

This particular embodiment allows for the processing of culture dishes positioned upright in the first storage carousel. The upper plate is used here for the rotational driving of closed culture dishes or lids, depending on the relevant processing station, while the lower plate is responsible for the rotational driving of the bases.

According to one specific feature, the stationary processing stations further comprise a culture dish opening station comprising:
a flap mounted so as to move between at least one position for holding the base attached to the lid and a release position allowing the base to be separated from the lid;
a manipulator mounted so as to move in translation between at least one position for picking up the base and a position for placing the base on the lower plate;

the flap comprising an opening for the introduction of the manipulator, said picking up position being assumed when the flap is in the base holding position after the manipulator has passed through the introduction opening of the flap.

According to one specific embodiment, the flap is mounted to rotate about a horizontal axis, allowing it to transition from the holding position to the release position, and vice versa.

According to one alternative embodiment, the flap is mounted so as to rotate about a vertical axis of an actuator which is configured to exert pressure on a pin arranged in an opening passing through the drive device, said pin exerting pressure on the lid when the flap is in the release position. The inventors discovered that this configuration greatly facilitates the opening of Petri dishes, the success rate of opening Petri dishes being greatly increased.

According to one specific feature, said at least one first and second transfer stations each comprise a manipulating member that is mounted so as to move in translation between at least one position for picking up the base from the lower drive plate, an intermediate position for securing the base to the lid at the upper drive plate, and a position for placing the base and the lid, secured together, in either the first or second storage carousel.

According to one specific feature, the stationary processing stations further comprise at least one culture dish identification station belonging to the group comprising: a front bar code reader identification station and a side bar code reader identification station, said upper sliding surface covering the lower face of the upper drive plate on the receiving station and said at least one identification station. This allows the machine to identify each culture dish being processed and associate it with the type allocated to it.

According to one specific embodiment, said at least one first transfer station is intended to transfer to the first carousel the culture dishes allocated a first type (for example, dishes tested negative for the presence of a specific microorganism) and said at least one second transfer station is intended to transfer to the second carousel the culture dishes allocated a second type (for example, dishes tested positive for the presence of a specific microorganism).

Thus, said drive device is designed to cooperate both with the first storage carousel for a dish delivered to said at least one transfer station and with the second storage carousel for a dish delivered to said at least one second transfer station. The sorting of first and second type dishes can therefore be performed entirely autonomously by the machine based on the analysis results.

According to one particularly interesting feature, the culture dish opening station is additionally designed to transfer to the first carousel the culture dishes allocated a third type related to a processing issue, the manipulator (M4) of said opening station being further mounted to move in translation between a position, among said picking up position and said placing position, and a dish transfer position in the second storage carousel.

Thus, the opening station of the machine serves two functions: a first function opening culture dishes that do not present any processing issues, and a second function of transferring problematic culture dishes to the second carousel. “Problematic” refers to a culture dish for which the bar code could not be read by said at least one identification station arranged upstream in the processing cycle, or a culture dish for which the lid could not be separated from its base at the opening station. Such an approach enables more thorough sorting of the culture dishes by setting aside those that present an issue in the processing cycle.

According to a second embodiment of the drive device, the culture dishes to be processed each being provided with a lid attached to a base and positioned in a so-called upside down position in the first carousel:
the upper housings are each provided with a through-hole dimensioned to allow the passage of a culture dish, the upper sliding surface extending partially under the upper drive plate to hold the culture dish closed in the upside down position in the upper housing on at least one of the processing stations;
the lower housings are each provided with a through-hole dimensioned to allow the passage of a culture dish, the lower sliding surface extending under the lower drive plate to support the base and hold the culture dish closed in the upside down position in the lower housing.

This alternative embodiment allows processing of Petri dishes arranged upside down in the first storage carousel. Thus, the invention provides the ability to carry out processing on culture dishes that can be positioned either upright or upside down, simply by swapping the double drive plate. The machine according to the invention is therefore particularly versatile.

In another embodiment of the invention, a method is proposed for processing culture dishes using an automated processing machine comprising a first storage carousel, a second storage carousel, and an operational unit designed to receive the culture dishes in continuous succession and to carry out a series of successive treatments on each of the culture dishes, the operational unit comprising a plurality of stationary processing stations arranged at constant intervals around an axis of rotation and a drive device for rotating the culture dishes relative to the processing stations, said drive device comprising housings for the culture dishes arranged at intervals corresponding to those of the processing stations, such that a rotation of the drive device about the second axis successively positions a given culture dish at each of the processing stations in order to carrying out the following steps, at least partially simultaneously:
receiving a culture dish to be processed from the first carousel;
analysing the contents of the received culture dish and allocating to said dish a specific type from at least two predefined types of dish based on the analysis results;
transferring the culture dish, the contents of which have been analysed, to either the first or second storage carousel, depending on the type allocated to said culture dish.

In another embodiment of the invention, a computer program product is proposed, comprising program code instructions for implementing the aforementioned method in any one of its various embodiments, when said program is executed on a computer.

In another embodiment of the invention, a non-transitory computer readable storage medium is proposed, storing an aforementioned computer program product.

### LIST OF THE FIGURES

Other features and advantages of the invention will become apparent upon reading the following description, given by way of illustrative and non-limiting example, and the appended drawings, wherein:

FIG. 1 is a schematic representation of an automated processing machine according to one specific embodiment of the invention;

FIG. 2 is a perspective view showing a Petri dish drive device forming part of the machine shown in FIG. 1;

FIG. 3 is an exploded view of the drive device shown in FIG. 2;

FIG. 4 is a cross-sectional view showing the structure of a housing of the drive mechanism;

FIGS. 5, 6, and 7 schematically and sequentially show the step of receiving a Petri dish within the operational unit according to the invention;

FIGS. 8, 9, 10, and 11 sequentially show the step of opening a Petri dish according to the invention;

FIG. 12 provides a simplified schematic representation of the principle of separation between the lid and the base of a Petri dish during the opening step according to the invention;

FIG. 13 schematically shows the step of analysing the contents of a Petri dish according to the invention;

FIG. 14 is a partial view of the machine highlighting two positive-type dish transfer stations according to the invention;

FIGS. 15, 16, 17, and 18 schematically and sequentially show the step of transferring a Petri dish to one of the storage carousels according to the invention;

FIG. 19 schematically shows the step of reading a front bar code according to the invention;

FIG. 20 schematically shows the step of reading a side bar code according to the invention;

FIG. 21 is an exploded view of a drive device according to an alternative embodiment of the invention;

FIG. 22 shows the simplified structure of a processing unit implementing the method according to one specific embodiment of the invention;

FIG. 23 is a partial view of the processing machine showing the principle of transferring a culture dish between the storage carousel and the drive device;

FIGS. 24A and 24B are two partial views of the machine showing the Petri dish opening station according to one variant of the invention;

FIGS. 25A, 25B, 25C, 25D and 25E sequentially show the step of opening a Petri dish according to the variant shown in FIGS. 24A and 24B.

### DETAILED DESCRIPTION OF THE INVENTION

In all of the figures of the present document, identical elements and steps are designated by the same reference numeral.

The invention is based on a comprehensive automation solution for all tasks involved in reading and sorting culture dishes. Its general principle involves equipping the machine for processing culture dishes with an operational unit with multiple processing stations, intended to receive the culture dishes in continuous succession and to automatically carry out a cycle of successive treatments on each of the received culture dishes. Each processing station has a specific role in the processing cycle as the culture dishes are brought to each of the stations, some of the processing stations being capable of being activated at least partially simultaneously. Such automation enables, inter alia, a reduction in the time taken to read and sort culture dishes, a decrease in the rate of errors and cross-contamination, and an increase in the processing rate.

The remainder of the document focuses more specifically on describing the invention in the context of processing Petri dishes intended for use in a food microbiology laboratory. Of course, the invention is not limited to this particular field of application, but is relevant for all types of culture containers requiring biological or microbiological analysis.

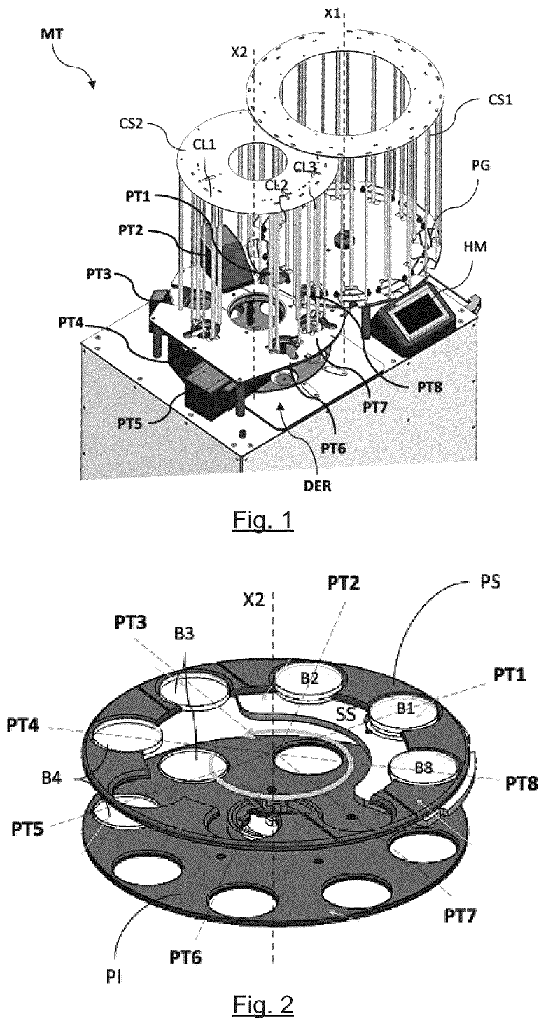

FIG. 1 shows an automated machine MT for processing Petri dishes according to one specific embodiment of the invention. The processing machine MT is shown empty, without Petri dishes, for the sake of clarity in the figure. It is a programmable machine that automatically carries out repetitive tasks at high speed and with precision, sorting Petri dishes based on their analysis results. The dishes testing positive for the presence of the specific microorganism(s) (listeria monocytogenes, Salmonella, Escherichia coli or Escherichia coli for example) are called “positive” dishes and those testing negative are called “negative”dishes.

The processing machine MT more specifically comprises a first storage carousel CS1 mounted for rotation about a first axis X1, a second storage carousel CS2 mounted fixed about a second axis X2, an operational unit positioned around the axis X2, and a human-machine interface HM. In this exemplary embodiment, the operational unit consists of eight stationary processing stations PT1-PT8 and a rotational drive device DER that enables the Petri dishes to be rotated about the axis X2 in relation to the processing stations PT1-PT8. All these elements are mounted on a frame, fitted with braked castors for example, to make it easier to move and set up the machine within the laboratory.

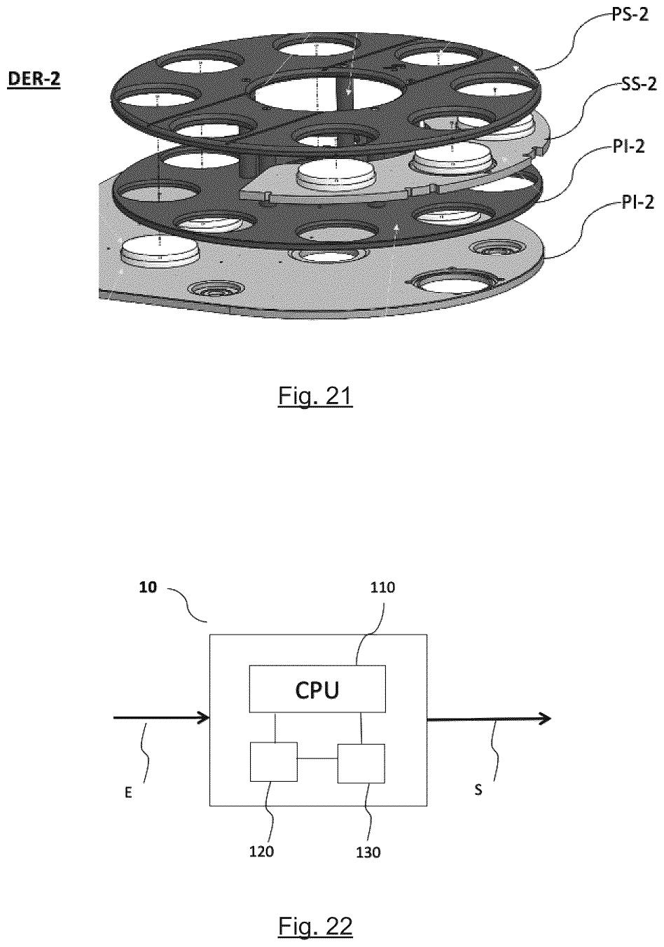

The various control equipment for these elements, in particular the drive motors for moving parts and the electronic components constituting the hardware unit controlling the machine, are installed inside the frame. This control unit (not shown in the figure) is electrically connected to the mobile storage carousel CS1, the various components of the drive device DER, and the HM interface, for the purpose of controlling these elements. The operating principle of this unit is detailed further in relation to FIG. 22.

### The Storage Carousel CS1

The storage carousel CS1 is designed to store Petri dishes to be processed. More specifically, for the purposes of the invention, this storage carousel CS1 functions both as a dispensing station for the dishes to be processed and as a receiving station for the dishes that tested negative for the presence of Listeria monocytogenes. The carousel CS1 has a substantially cylindrical structure made up of two horizontal plates that face each other and are connected by trios of vertical rods evenly distributed around the periphery of the structure along the axis X1. Each trio of rods, consisting of a rigid rod and two flexible rods arranged, for example, in a triangular formation, is designed to receive a column of stacked Petri dishes. The upper part of the trio of rods is made from a more flexible material than the rest to facilitate their elastic deformation when inserting the Petri dishes in batches. Alternatively, the entire rod is made of a flexible material suitable for this function. The carousel CS1 shown here has twelve storage columns angularly distributed evenly around the periphery of the structure. Each column can contain at least forty Petri dishes, meaning that the carousel CS1 can contain at least four hundred and eighty dishes. The lower plate of the carousel rests on a sliding plate PG and is traversed, at each of the storage columns, by an opening allowing a Petri dish to pass and slide on the sliding plate PG. The carousel CS1 cooperates with a rotational drive system (for example a stepper motor mechanically connected to a hub integral with the lower plate) to make the carousel rotate about the axis X1 relative to the sliding plate PG in increments corresponding to 1/12th of a turn, i.e. angular increments of 2π/12 in the anticlockwise direction. It should be noted that the sliding plate PG also comprises two openings, one allowing the passage of the dishes to be processed from the carousel CS1 (from a storage column known as a “dispenser”) along the vertical axis XD to the operational unit, and the other allowing the passage of the negative dishes from the operational unit to the carousel CS1 (in a storage column known as a “receiver”) along the vertical axis XR.

### The Storage Carousel CS2

The storage carousel CS2 is designed to store Petri dishes that have tested positive for the presence of Listeria monocytogenes. Unlike the storage carousel CS1, which is mounted for rotation, the carousel CS2 is fixed about the axis X2. The carousel CS2 is made up of two horizontal plates that face each other and are connected by set of vertical rods evenly distributed around the periphery of the structure, forming the columns of the positive Petri dishes of the machine. The vertical rods are flexible at the top to allow them to spread apart, making it easier to remove Petri dishes from a stack of dishes. The carousel CS2 shown here has three receiving columns CL1-CL3 distributed around the axis X2 associated respectively with the processing stations PT4, PT6 and PT7 of the operational unit. The receiving column CL1 is designed to hold “problematic” dishes. The receiving columns CL2-CL3 are designed to accommodate the “positive” Petri dishes. The lower plate of the carousel features an opening at each of the receiving columns, allowing Petri dishes to pass from the operational unit to the carousel CS2.

### The Operational Unit With Multiple Processing Stations

The operational unit is designed to receive Petri dishes in continuous succession from the storage carousel CS1 and to carry out a series of successive treatments on each of the received culture dishes, using the stationary processing stations PT1-PT8 and the drive device DER.

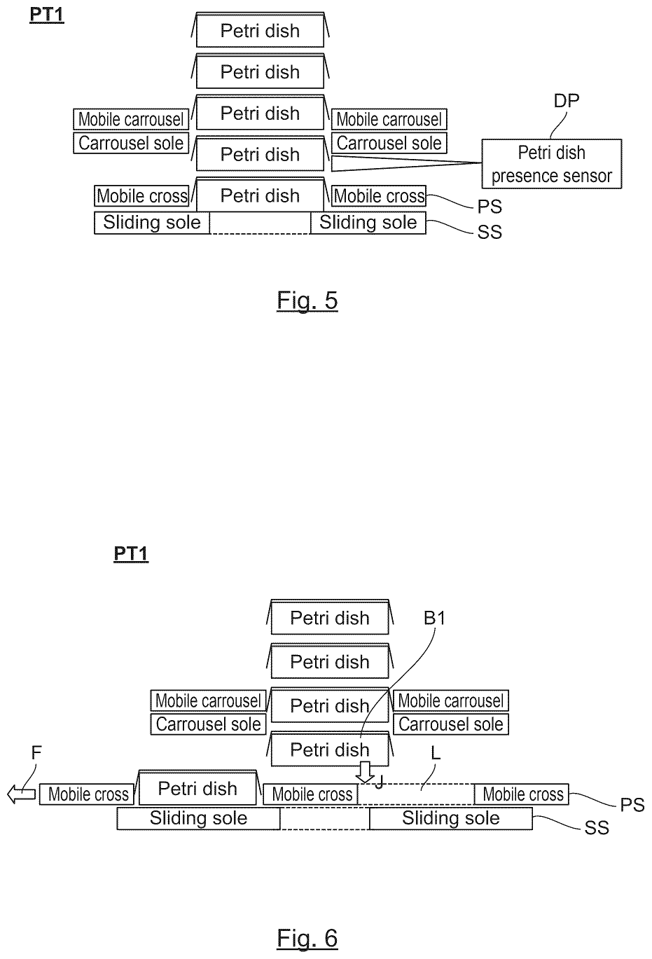

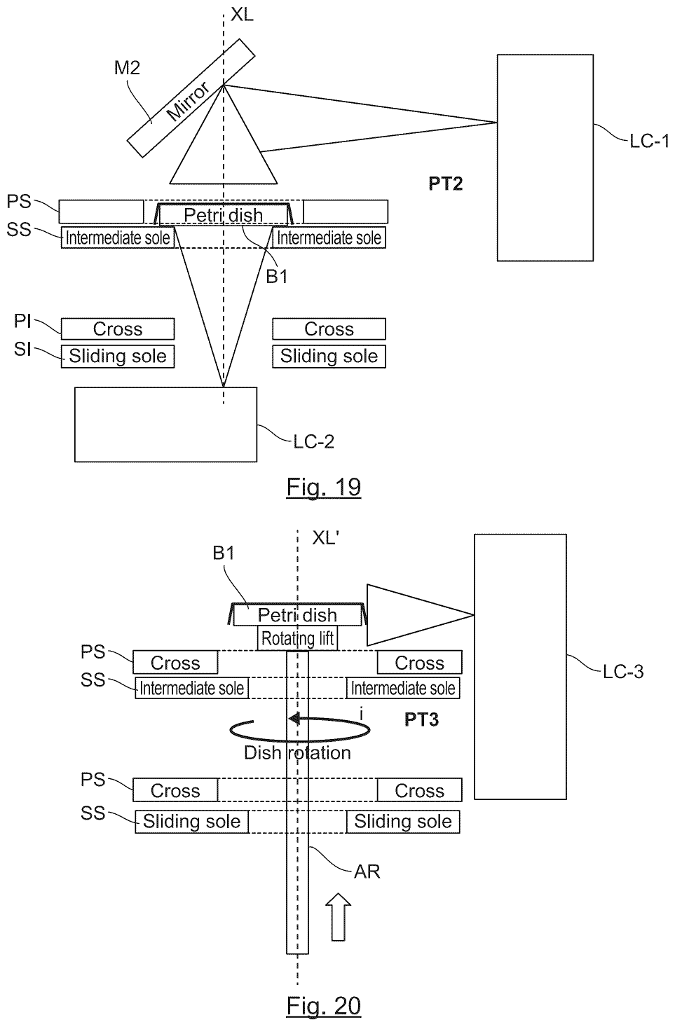

The operational unit comprises, for a given Petri dish:
a receiving station PT1 designed to receive the Petri dish from the storage carousel CS1 (operating principle described later in relation to FIGS. 5 to 7);
a front bar code reading station PT2 designed to read a bar code that may be affixed to the lid or the base of the Petri dish (operating principle described later in relation to FIG. 19);
a side bar code reading station PT3 designed to read the bar code that may be affixed to the side of the Petri dish (operating principle described later in relation to FIG. 20);
a culture dish opening station PT4 intended to separate the lid from the base of the Petri dish (operating principle described later in relation to FIGS. 8 to 12) and to transfer, if necessary, the “problematic” Petri dishes;
an analysis and allocation station PT5 designed to analyse the contents of the Petri dish and allocate it a specific type from at least two predefined dish types (positive or negative) based on the analysis results (operating principle described later in relation to FIG. 13);

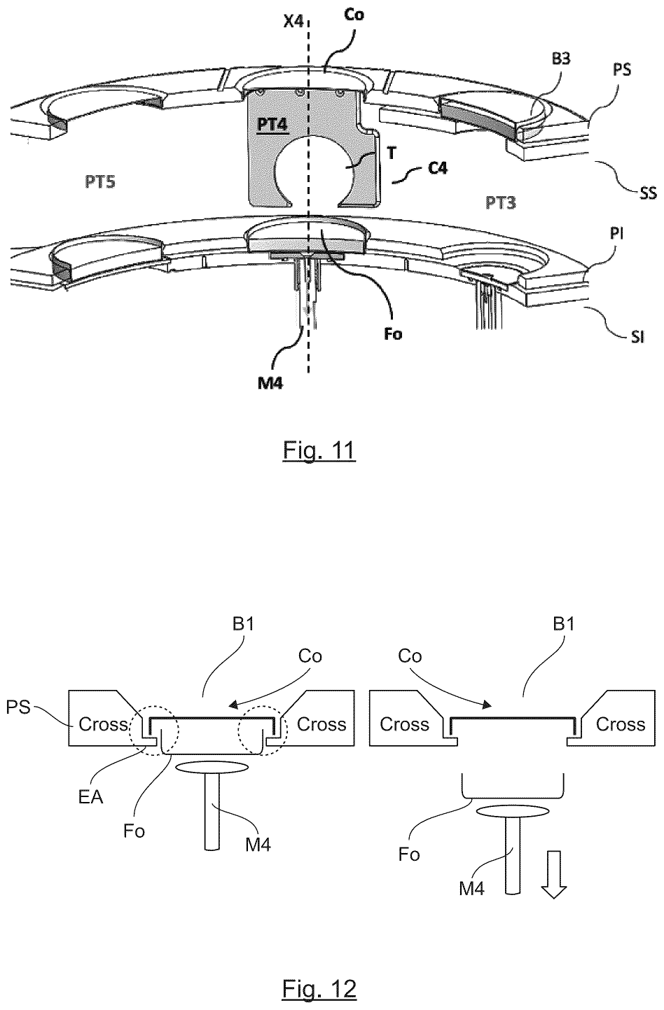

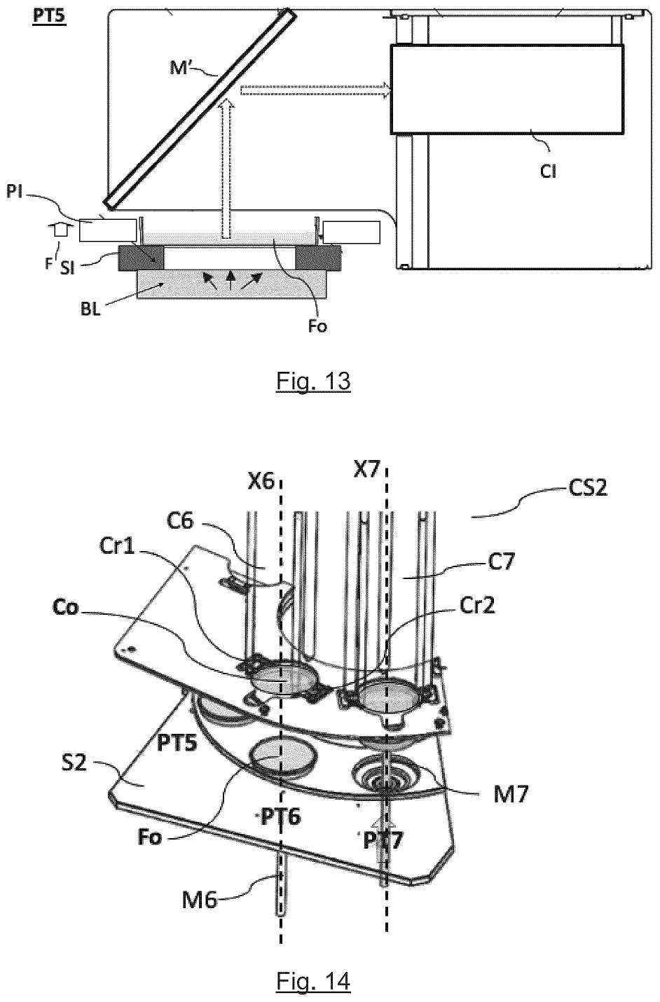

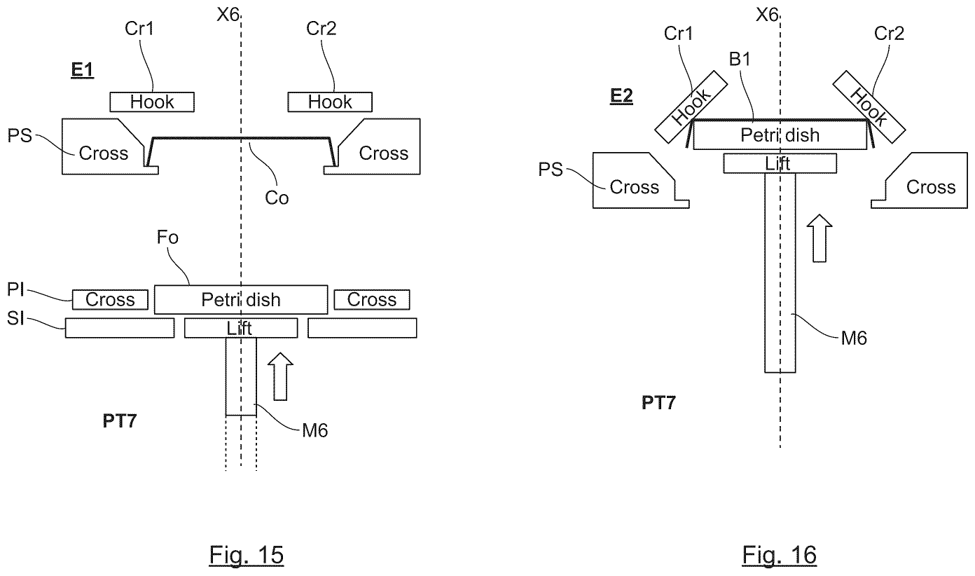

two positive dish transfer stations PT6-PT7 each intended to transfer the Petri dish to the storage carousel CS2 in the event of a positive result for the presence of Listeria monocytogenes allocated to the station PT5 (operating principle described later in relation to FIG. 14);
a negative dish transfer station PT8 intended to transfer the Petri dish to the storage carousel CS1 in the event of a negative result for the presence of Listeria monocytogenes allocated to the station PT5 (operating principle described later in relation to FIGS. 15 to 18).

The processing stations PT1-PT8 are referred to as “stationary” because they are securely mounted on the frame of the machine MT in relation to the axis X2. The stations PT1-PT8 are evenly distributed around the axis X2 in increments substantially equal to ⅛th of a turn, i.e. angular increments of 2π/8.

To improve processing rate, the processing stations PT1-PT8 and the control unit are configured, in this exemplary embodiment, to allow the processing stations to be activated at least partially simultaneously. Indeed, the processing stations with substantially identical processing times are activated simultaneously for their entire duration, whereas the processing stations with different times are activated partially simultaneously (typically, a short processing station will start after a long processing station has begun or will finish before it ends).

The operational unit further comprises a drive device DER for rotating Petri dishes about the axis X2. Such a device comprises eight Petri dish housings arranged at intervals corresponding to those of the processing stations PT1-PT8, specifically at angular increments of 2π/8, such that a rotation of the drive device DER about the axis X2 successively positions the Petri dish at each of the processing stations PT1-PT8. To achieve this, the drive device DER cooperates with a rotational drive system (described below) that enables the Petri dishes (positioned in the housings) to undergo a discontinuous rotational movement, specifically rotating in steps at angular increments of 2π/8 relative to the lower plate of the carousel CS2.

It should be noted that the processing machine MT has as many Petri dishes being processed as there are workstations within the operational unit. All workstations operate continuously, except when the drive device DER is rotating to transfer dishes from one station to another.

The structure of the rotational drive device according to the invention is now described in more detail, in relation to FIGS. 2 to 4.

First of all, it is important to note that Petri dishes are small containers made of optically transparent plastic, consisting of a base and a lid. Each of these parts has a side wall of varying diameters, with the wall of the lid overlapping that of the base when the dish is closed. This set-up is relatively frictionless, allowing the lid to be removed from the base of the dish effortlessly (this will also be referred to as the “separation” of the base from the lid).

In this exemplary embodiment, the drive device DER comprises a double rotating drive plate made up of an upper drive plate PS and a lower drive plate PI. The drive plates PS and PI are arranged parallel to each other and are interconnected by linking elements, such as spacers BL. The drive plates PS and PI have a substantially annular disc shape. They are mounted to rotate about the X2 axis using a rotational drive system, such as a stepper motor mechanically connected to a central hub that is integral to the plate PI and/or plate PS, aligned with the axis X2. The stepper motor is configured to drive the double plate in a stepwise rotational movement, in angular increments of 2π/8, relative to the upper sliding surfaces SS and lower sliding surfaces SI of the device. The upper sliding surface SS is securely mounted on the frame in relation to the axis X2 and partially extends over the lower face of the upper plate SS to ensure that the base remains attached to the lid of the Petri dishes on the stations PT1, PT2 and PT3. The lower sliding surface SI is securely mounted on the frame in relation to the axis X2 and extends over the lower face of the lower plate PI. The sliding surfaces SS and SI may also be equipped with through-holes depending on the relevant processing station, dimensioned to allow the passage of a manipulator (in the case of stations PT4, PT6, PT7, and PT8) or a backlighting light beam (in the case of station PT5), while ensuring the integrity of the base of the dish.

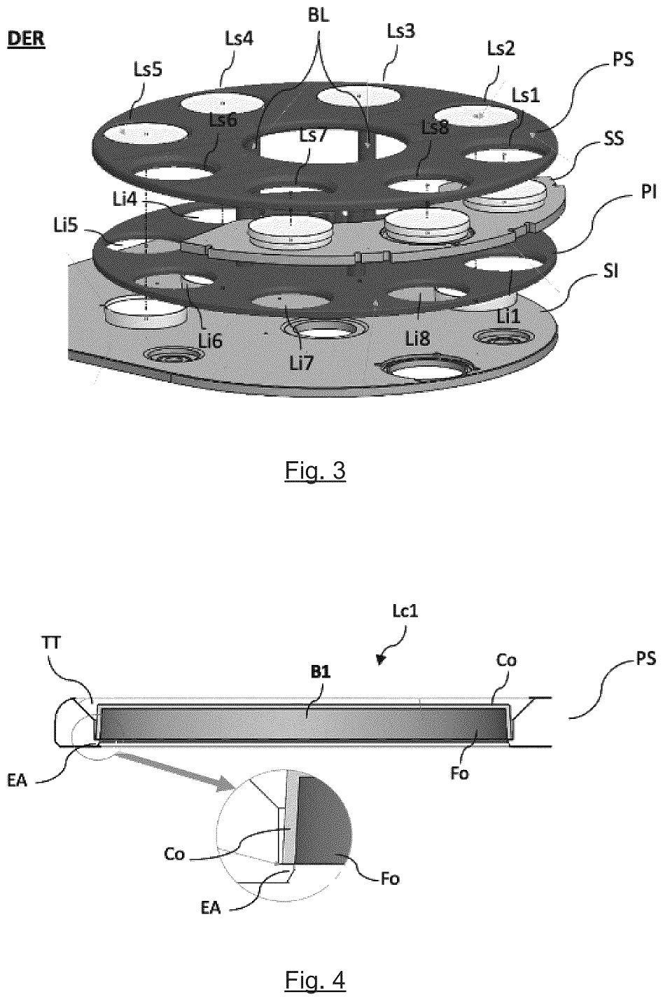

The upper plate PS comprises a series of eight upper housings, labelled Ls1-Ls8 in FIG. 3, evenly distributed at angular increments of 2π/8 around the periphery of the upper plate PS about the axis X2. The lower plate PI comprises a series of eight lower housings, labelled Li1-Li8, evenly distributed at angular intervals of 2π/8 around the periphery of the plate about the axis X2, with their vertical axes aligning with those of the upper housings Ls1-Ls8. Thus, thanks to the presence of the spacers, the housings Ls1-Ls8 on the upper plate and the housings Li1-Li8 on the lower plate are arranged facing each other in pairs, forming upper-lower housing pairs that remain coupled throughout the processing cycle.

The upper housings Ls1-Ls8 each feature a through-hole dimensioned to allow the passage of a Petri dish, ending with an annular shoulder EA, which is designed to hold the lid of the dish in the upper housing while allowing the base of the dish to pass through by gravity (in the absence of a sliding surface). FIG. 4 shows, for example, the detail of the upper housing Ls1 that houses a Petri dish B1. The base of the dish is labelled Fo and the lid of the dish, Co. In this specific embodiment, the Petri dish B1 is positioned upright in the housing Ls1. The latter comprises a through-hole TT including a first portion of conical shape (leading to a gradual cross-section reduction), a second cylindrical portion, and terminating with a lid retaining annular shoulder EA.

The lower housings Li1-Li8 each feature a through-hole dimensioned to allow the passage of the base of the Petri dishes through the lower drive plate PI, the base of the dishes being supported on the sliding surface SI. These holes also allow the passage of a manipulator (for stations PT3, PT4, PT6, PT7, and PT8) or the passage of a rear light beam (for station PT5).

In this specific embodiment, the function of the drive plate PS is therefore, in the presence of the sliding surface SS, to deliver a complete Petri dish (base and lid assembly) to each of the processing stations PT1, PT2, and PT8 (for dishes B1, B2, and B8 in FIG. 2), and, in the presence of the sliding surface SS, to deliver only the dish lid to each of the processing stations PT3, PT4, PT5, PT6, and PT7 (for dishes B3 and B4). While the function of the drive plate PI is to support the base of the Petri dishes and transport it to each of the processing stations PT3, PT4, PT5, PT6, and PT7 (for Petri dishes B3 and B4 for example). It should be noted that transporting closed Petri dishes when processing permits reduces the risk of cross-contamination.

The paired alignment of the upper housings Ls1-Ls8 and lower housings Li1-Li8 ensures that, from one processing station to another and throughout the processing cycle of a Petri dish, the lid remains aligned with the base of the dish, even in the event of a stoppage of the machine MT. This helps to minimise the risk of swapping the lid and/or base between different Petri dishes, thereby ensuring the traceability of the analysed sample (tracking provided by the bar code affixed to the base and/or lid of the dish).

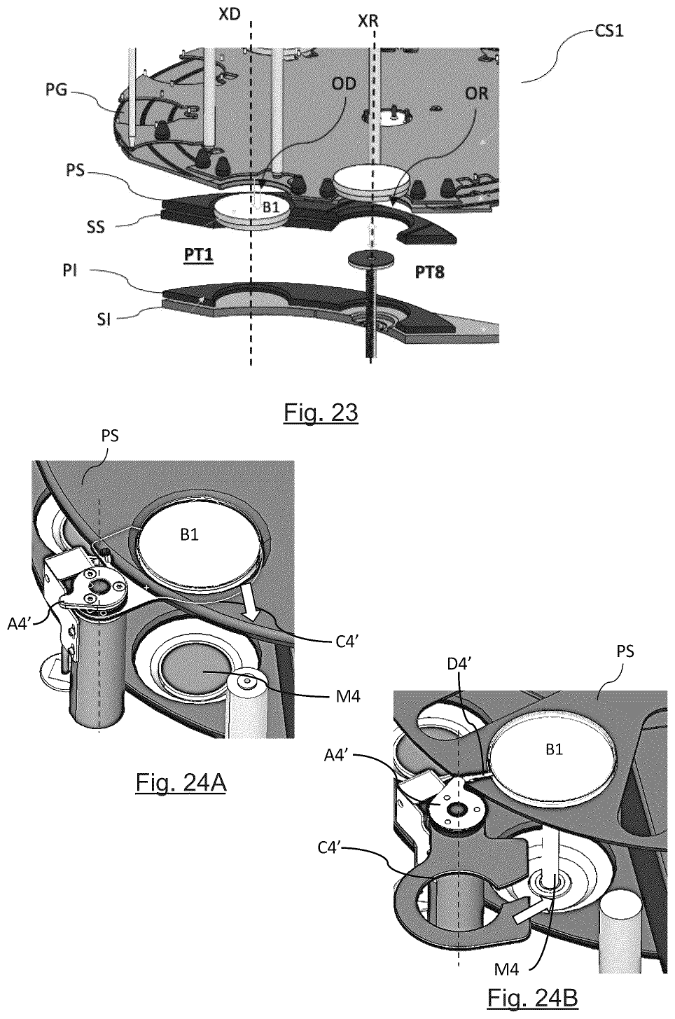

It should be noted that the carousel CS1 and the double drive plate partially overlap at certain points along their respective peripheries, allowing for the alignment of the dispensing opening OD-at the receiving station PT1—with one of the housing pairs of the drive plate, and the alignment of the receiving opening OR-at the transfer station PT8-with another housing pair of the drive plate (as shown in more detail in FIG. 23). In other words, the carousel CS1 and the double drive plate PS-PI are partially interlocked to align the positions of their openings during their respective rotations.

### The Human-Machine Interface HM

The HM interface is the control panel that allows the user to communicate with the machine MT, issuing commands for the desired processing cycle and responding to instruction requests from the microprocessor. These instructions specifically address: the number of dishes to be processed, the type of agar present in the Petri dishes, the imaging parameters, and the nature of the specific microorganisms. The HM interface allows the invention to be used in automatic or semi-manual mode. Access to automatic mode allows processing of Petri dishes to begin once they have been loaded into the carousel CS1. Semi-manual mode provides access to all processing stations and allows them to operate independently of each other.

### Exemplary Machine Operation

In relation to FIGS. 5 to 20, the operation of the machine MT will be set out below, step by step, throughout a complete processing cycle for a specific Petri dish. These steps are carried out by a processing unit of the machine (the principle of which is detailed later in relation to FIG. 22).

After placing the Petri dishes in the storage carousel CS1, the operator starts a processing cycle through the HM interface. Care should be taken to maintain at least one empty storage column available for the reception and stacking of positive Petri dishes from the operational unit. Optionally, the Petri dishes can be inserted into the storage columns, not after but before the actual installation of the carousels CS1 and CS2 on the frame of the machine.

Petri dishes are considered to be filled with a food sample for which the presence of Listeria monocytogenes is tested.

At the start of the operation, to compensate for any play between the parts, the carousel CS1 and the drive device are briefly rotated to ensure perfect centring of the sliding plate PG and the carousel CS1 along the axis X1, as well as perfect centring of the double plate PS-PI along the axis X2. Sensors are provided to ensure the correct positioning of the carousels and the drive device during start-up and throughout the processing cycle.

Step 1: Transfer of the Petri Dish to the Operational Unit (FIGS. 5-7, 23)

The first step is to transfer the Petri dish from the carousel CS1 where it is stored to the drive device DER, at the workstation PT1. To achieve this, the carousel CS1 and the drive device DER are each rotated until the vertical axis associated with the storage column and that associated with the pair of housings of the double plate align with the dispensing opening axis XD. Once the openings are placed opposite each other along the axis XD, the Petri dish B1 located below the storage column is automatically transferred, under the effect of gravitational forces acting on it, into the housing L of the upper plate PS through the dispensing opening OD. The base of the dish then rests on the sliding surface SS.

The presence of chamfers on the outer edges of the openings of the carousel CS1 and the drive plate PS makes it easier to transfer the dish (gradual transition) by minimising the risk of snagging, particularly in the space between the storage carousel and the drive plate.

The presence of the sliding surface SS serves as a support for the Petri dish (or at least the base of the dish) in the upper housing of the plate PS1 and facilitates its sliding when the upper plate PS is rotated about the axis X2 in relation to the sliding surface SS.

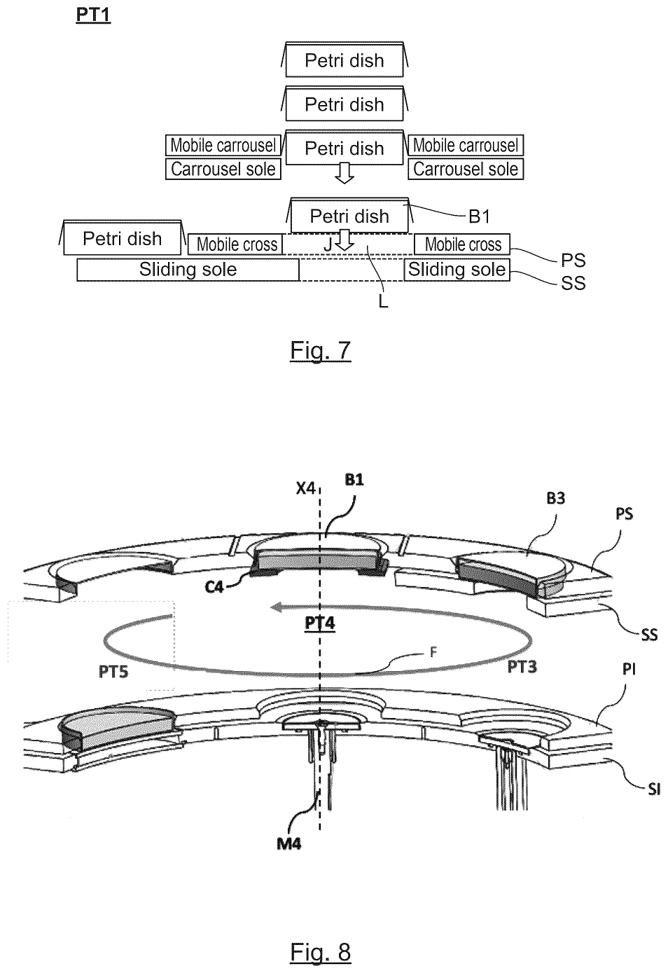

A presence detector DP, connected to the processing unit, is provided to detect the presence of a Petri dish in the opening OD waiting to be transferred to the next housing of the upper plate. The presence of a dish detected by the detector DP triggers the movement of the double plate to rotate by angular increments of 2π/8 about X2 in the anticlockwise direction (as indicated by the arrow F in FIG. 6), resulting in the displacement of the Petri dish B1 from the station PT1 to the station PT2, along with the other dishes already present in the drive device DER to the subsequent workstations. When the rotation is complete, the next Petri dish in the storage column is automatically transferred to the next housing of the drive plate, and so on. The different Petri dishes stored therefore arrive successively at the station PT1 (movement shown by the arrow J in FIGS. 6 and 7).

Thus, the movement of the double drive plate, synchronised with the transfer of a new Petri dish to the operational unit, enables the automation of moving Petri dishes from one workstation to another, from their initial storage area to their final storage area.

Alternatively or in addition, a manipulating member that is mobile in translation along the axis XD may be provided to facilitate the transfer of Petri dishes from the carousel CS1 to the drive device DER.

When the last dish from a storage column of the carousel CS1 is transferred to the operational unit, the absence of a dish detected by the detector DP causes the carousel CS1 to be displaced by angular increments of 2π/12 about the axis X1, in order to position a new stack of dishes above the dispensing opening OD, and thereby continue the continuous transfer of the culture dishes still stored in the carousel CS1 to the operational unit. If the new storage column located above the dispensing opening OD is empty, the drive system of the carousel CS1 is reactivated by angular increments of 2π/12 about the axis X1 to process a new stack of dishes, and so on until all the dispensing columns have been processed.

The double plate is then activated by the control unit to move the Petri dish by angular increments of 2π/8 about the axis X2, in order to transfer it from the station PT1 to the station PT2.

Step 2: Front Bar Code Scanning (FIG. 19)

The second step is to read the bar code located on the lid or base of the Petri dish. This second step is carried out when the Petri dish is positioned at the station PT2.

The station PT2 comprises a first bar code reader LC-1, arranged at the periphery of the drive device DER and perpendicular to the axis of the housing XL, cooperating with a reflective mirror M, arranged above the plate PS and in the axis XL, to enable a front optical reading (full field or by scanning) of the bar code located on the lid of the Petri dish. Alternatively or additionally, the station PT2 is also equipped with a second bar code reader LC-2 positioned beneath the drive device DER, designed to enable rear optical reading (either full field or by scanning) of the bar code located on the base of the Petri dish.

The bar code readers LC-1 and LC-2 of the machine are activated when the double plate has completed its rotation of 2π/8. The function of this workstation is to allow bar code reading and identification of the sample contained in the Petri dish regardless of the position of the bar code on the dish. The data obtained from the bar code scan is stored in a storage memory of the machine. They allow the Petri dish to be monitored throughout processing on the different workstations of the operational unit.

The double plate is then activated by the control unit to move the Petri dish by angular increments of 2π/8 about the axis X2, in order to transfer it to the station PT3.

Step 3: Side Bar Code Scanning (FIG. 20)

The third step is to read the bar code located on the lateral surface of the Petri dish. This third step is carried out when the Petri dish is positioned at the station PT3.

The station PT3 comprises a side bar code reader LC-3, arranged at the periphery of the drive device DER and perpendicular to the axis of the housing XL′, along with a manipulator M1 that is mobile in translation along the axis XL′ and cooperating with the reader LC-3 to facilitate the optical reading of the bar code located on the edge of the Petri dish. The manipulator M3 is mobile in translation between a low holding position on the plate PS and a high reading position by the reader LC-3. The manipulator M3 is additionally mounted to rotate about a pivot axis of 360 degrees (movement indicated by arrow i), allowing the reader LC-3 to scan the entire surface of the edge of the Petri dish when the manipulator M3 is in the reading position to ensure that the bar code is read.

The double plate is then activated by the control unit to move the Petri dish by angular increments of 2π/8 about the axis X2, in order to transfer it to the station PT4.

Step 4: Opening the Petri Dish or Transfer (FIGS. 8 to 12)

The fourth step is to separate the lid from the base of the Petri dish. This fourth step is performed when the Petri dish is positioned at the station PT4.

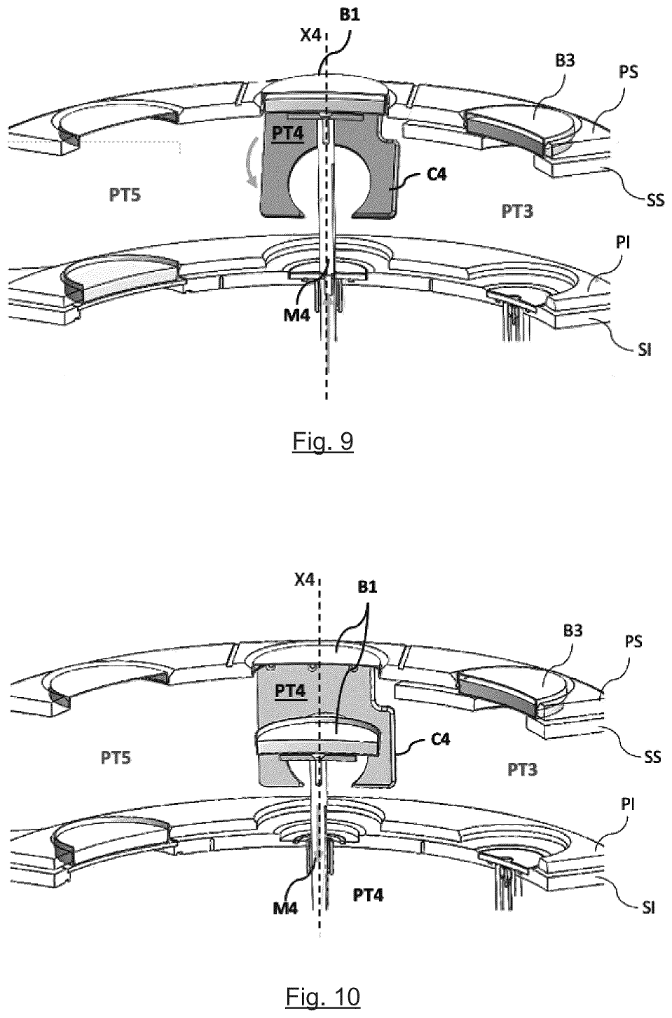

The station PT4 comprises a flap C4 mounted so as to move between a position for holding the base attached to the lid (FIG. 8) and a release position allowing the base to be separated from the lid (FIGS. 10-11). The station PT4 further comprises a manipulator M4 mounted so as to move in translation along the axis X4 between a position for picking up the base (FIG. 9) and a position for placing the base (FIG. 11) on the lower plate PI. The flap C4 comprises an opening T for the introduction of the manipulator M4. The picking up position is assumed when the flap C4 is in the base holding position after the manipulator M4 has passed through the introduction opening T of the flap C4. The manipulator M4 cooperates with a drive motor (not shown), which is controlled by the control unit and, when activated, facilitates the vertical movement thereof between the picking up position and the position for placing the base.

As shown in FIG. 8, when the Petri dish arrives, the movable flap C4 is positioned horizontally, in line with the sliding surface SS (i.e. extending beneath the lower surface of the upper plate PS) to ensure that the base Fo is securely attached to the lid Co (with only the lid Co actually being held in place by the upper housing Ls1 of the double drive plate). At this stage, the function of the flap is to prevent the base Fo of the dish from falling, by gravity, due to the absence of the sliding surface at the station PT4. The manipulator M4, positioned along the axis of the introduction opening T of the flap, is situated in the lower position beneath the lower plate PI, extending from the sliding surface SI.

Then, as shown in FIG. 9, the manipulator M4 is activated by the control unit to cause its displacement by vertical translation until it makes contact with the Petri dish B1. At this moment, the manipulator M4 is in the high position and holds the base Fo of the Petri dish (picking up position). The movable flap C4 is activated by the control unit to transition from its holding position (horizontal position) to its release position of the base of the dish (vertical position) through a rotational movement about a horizontal axis. The base Fo can then be separated from the lid Co of the dish, the base Fo now resting on the manipulator M4.

Finally, as shown in FIGS. 10 and 11, the manipulator M4 is once again activated by the control unit to cause it to move from its picking up position to its position for placing the base Fo on the lower plate of the drive device. The Petri dish opens naturally due to gravity, as nothing keeps the base Fo of the dish attached to its lid Co, which rests on the edges of the upper housing Ls1. This separation phase is shown schematically in FIG. 12. Once the placing position has been assumed by the manipulator M4, the base Fo rests in the lower housing Li1 of the double drive plate, while the lid Co of the dish is held in the upper housing Ls1 whose vertical axis coincides with that of the lower housing Li1.

At this stage, the operational unit therefore makes it possible to separate the base of the lid (containing the sample to be analysed) of the Petri dish, while maintaining the pairing of these two elements throughout the processing cycle thanks to the double drive plate. In fact, even if the machine stops unexpectedly, it is still easy to reassemble the Petri dish without the risk of mixing up the dish bases and lids. This reduces the risk of errors in processing results.

Furthermore, the station PT4 is configured to transfer the Petri dish when it is deemed problematic by the processing unit. “Problematic” means a Petri dish that could not be identified at the station PT2 and/or PT3 or a Petri dish that could not be opened at the station PT4. For this purpose, the manipulator M4 is further mounted so as to move in translation along the axis X4 between the aforementioned picking up position or the aforementioned placing position and a transfer position in the receiving column CL1. The picking up position of the manipulator M4 constitutes an intermediate position between a low placing position and a high transfer position. In the case of an unidentified Petri dish, for example, the flap is not activated and remains in its original position, keeping the base secured to the lid. The transfer mechanism for a “problematic” Petri dish is then initiated (the principle of which is identical to that described later for the station PT6).

The processing unit is configured to activate the manipulator M4 and/or the flap C4 in accordance with the principles outlined above, depending on the identification result obtained earlier for the Petri dish. The double plate is then activated by the control unit to move the Petri dish (base Fo and lid Co) by angular increments of 2π/8 about the axis X2, in order to transfer it to the station PT5. The flap C4 then returns to its initial holding position.

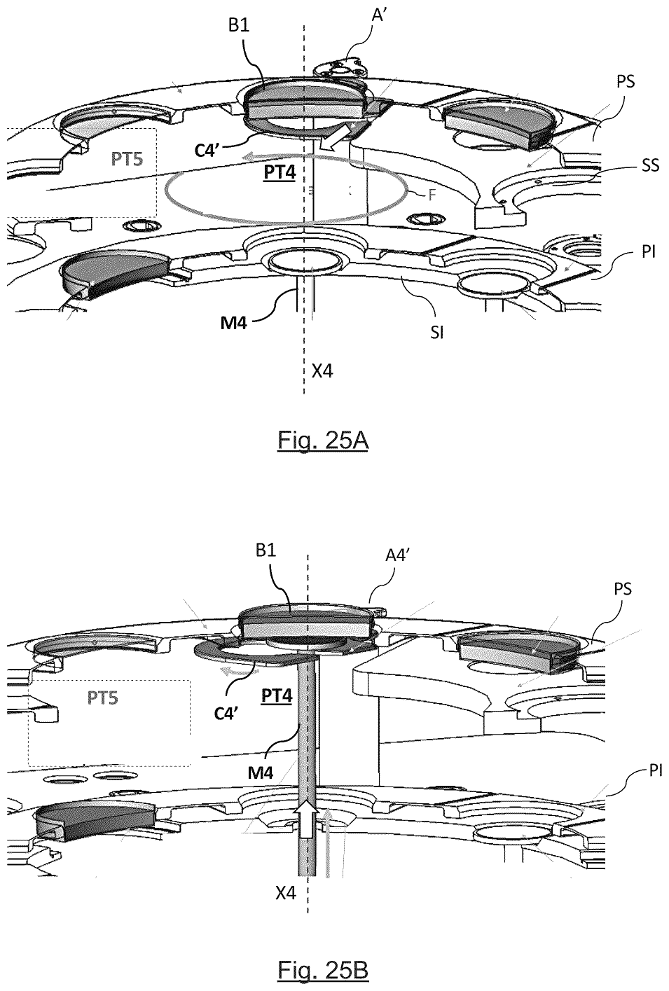

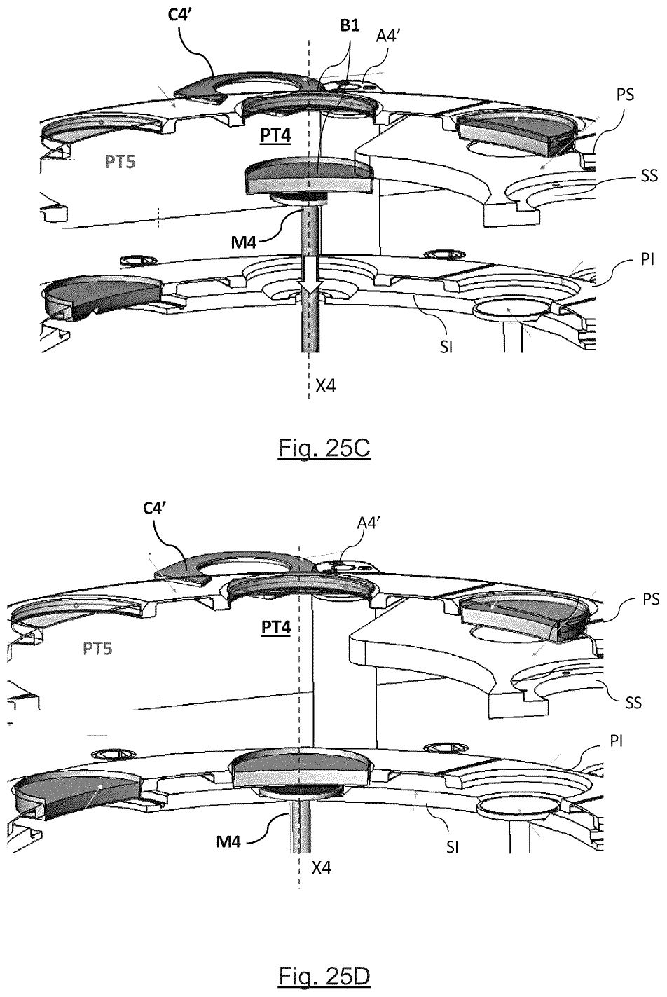

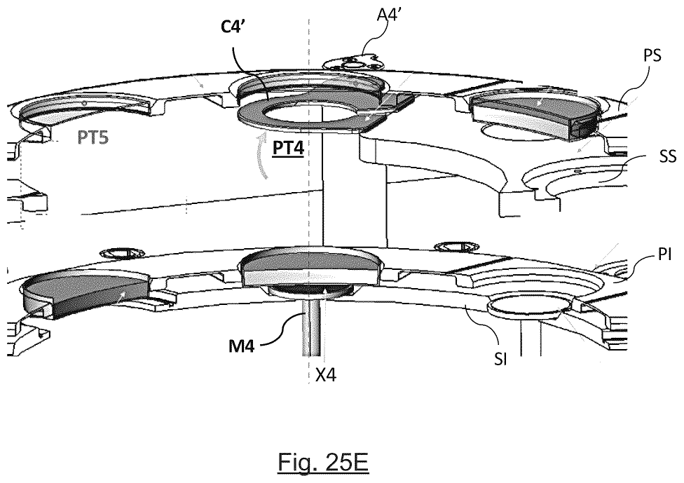

An alternative embodiment of step 4 is described below, in relation to FIGS. 24A, 24B, 25A to 25E. This facilitates the opening of Petri dishes, especially in situations where the lid is stuck to the base of the Petri dish, making it difficult to separate them. Unlike the exemplary embodiment described above (in relation to FIGS. 8 to 12), the flap C4′ is mounted so as to move between two horizontal positions: a position for keeping the base secured to the lid (FIG. 24A) and a release position allowing the base to be separated from the lid (FIG. 24B). The flap C4′ comprises an opening for the introduction of the manipulator M4. The picking up position is assumed when the flap C4′ is in the base holding position after the manipulator M4 has passed through the introduction opening T.

When the Petri dish B1 arrives, the movable flap C4′ is positioned horizontally, in line with the sliding surface SS extending beneath the lower surface of the upper plate PS to ensure that the base is securely attached to the lid (FIG. 25A). At this stage, the function of the flap is to prevent the base of the dish from falling, by gravity, due to the absence of the sliding surface at the station PT4. The manipulator M4, positioned along the axis of the introduction opening of the flap, is situated in the lower position beneath the lower plate PI.

The manipulator M4 is activated by the control unit to cause its displacement by vertical translation until it makes contact with the Petri dish. At this moment, the manipulator M4 is in the high position and holds the base of the Petri dish (picking up position-FIG. 25B). The flap C4′ is activated by the control unit to transition from its holding position to its release position of the base of the dish through a rotational movement around a vertical axis, which moves the flap outside the rotational drive device, as shown in FIG. 24B. An actuator A4′ is attached to the vertical axis of the flap and mounted so that it can rotate around this same axis and is configured to exert pressure on a pin (or finger) D4′ arranged in an opening passing through the drive device from its peripheral edge to the relevant housing. The pin D4′ is arranged to be above each of the edges of the upper housings Ls of the drive device, as shown in FIG. 24B. In the release position, the pressure applied to pin D4′ exerts force on the lid of the Petri dish: the lid becomes trapped between the edge of the upper housing Ls and pin D4′, elastically deforming the lid and creating an airflow into the Petri dish. This airflow will help to separate the lid from the base of the Petri dish in addition to the deformation of the lid. The pressure of the pin on the lid is maintained for a predefined time controlled by the control unit, after which the flap C4′ performs a slight rotational movement to the right, at the command of the control unit. This slight rotational movement of the flap relieves the pressure of the pin D4′ on the lid, which is then no longer trapped in the upper housing of the rotational drive device. Thanks to this dual pressure, the base can then be easily detached from the lid of the dish, with the base now resting freely on the manipulator M4. This alternative embodiment is particularly well suited in cases where difficulties may arise in detaching the lid.

The manipulator M4 is once again activated by the control unit to cause it to move from its picking up position to its position for placing the base on the lower plate of the drive device (FIG. 25C). The Petri dish opens naturally due to gravity, as nothing keeps the base of the dish attached to its lid, which rests on the edges of the upper housing Ls1. Once the placing position has been assumed by the manipulator M4, the base rests in the lower housing Li1 of the double drive plate, while the lid of the dish is held in the upper housing Ls 1 (FIG. 25D).

Then the flap C4′ is once again activated by the control unit to return to its initial position in the drive device through a rotational movement around the vertical axis of the actuator A4′ (FIG. 25E).

Step 5: Analysis of the Contents of the Petri Dish (FIG. 13)

The fifth step involves analysing the food sample contained in the Petri dish in order to assign a positive or negative type depending on the presence or absence of the specific pathogens. This fifth step is performed when the Petri dish is positioned at the station PT5.

The station PT5 is equipped with an imaging module comprising a light source BL, an image sensor CI and an optically reflective mirror M′. These elements are placed in an optically opaque enclosure to block any optical phenomena that could interfere with the analysis. The processing unit is electrically connected to the light source BL and the image sensor CI for the purpose of controlling these elements and processing the results. The light source BL, the image sensor CI, and the reflective mirror M′ are positioned to facilitate an analysis by light transmission through the base of the dish Fo. The light source BL is arranged in a backlight configuration relative to the sample. In this example, the light source BL is a light-emitting diode that emits white light with an illuminance of 800 to 6500 lux. The image sensor CI is designed to detect the light intensity received from the sample contained in the base of the dish Fo and convert it into an electrical signal for the control unit. In this example, the image sensor CI is a CMOS sensor.

The processing unit is configured to control, via the control unit, the imaging device so as to enable the acquisition of an image or a series of images of the sample, the analysis of the acquired images and the allocation of the given dish type according to the analysis results.

The analysis phase consists of the search, by image analysis, for the presence of Listeria monocytogenes bacteria in the sample. If the analysis result is positive for the presence of the specific microorganism, the Petri dish B1 is classified as “positive”. If the analysis result is negative, the Petri dish B1 is classified as “negative”. If the analysis result is inconclusive, the Petri dish B1 is also classified as “positive”.

Furthermore, it should be noted that if the Petri dish B1 could not be identified at the reading stations PT2 or PT3, the Petri dish B1 is classified as “problematic”. If the Petri dish B1 could not be opened at the opening stations PT4, it is also classified as “problematic”. The Petri dish is sorted by the machine based on the type of dish assigned to it by the processing unit.

According to one specific embodiment, the analysis phase further comprises the detection of the presence of colonies of microorganisms and the determination of the number of colonies present in the sample. Thus, each given sample identified by its bar code is assigned a dish type along with information about the number of colonies detected. This data is stored in the memory of the processing unit.

Of course, various methods known to those skilled in the art of detecting or recognising objects in an image may be used to proceed with image analysis and searching for colonies of microorganisms, such as a learning method based on an artificial intelligence process (for example: deep learning, machine learning, supervised learning) or a method based on the application of a predefined digital filter, image segmentation, texture recognition, or a method of self-correlation of image characteristics. A person skilled in the art, familiar with these methods, is able to tailor the algorithm used at this stage of the process, particularly based on the nature of the analysis to be conducted and the desired rates of false positives or false negatives.

Of course, the imaging module may be configured in other ways and comprise additional light sources to optimise illumination and light effects to facilitate the search for microorganisms.

The double plate is then activated by the control unit to move the Petri dish (base Fo and lid Co separated) by angular increments of 2π/8 about the axis X2, in order to transfer it to the station PT6.

Step 6: Transfer of “Positive” Dishes (FIGS. 14 to 18)

This sixth step involves reassembling the Petri dish and transferring it to the storage carousel CS2, if the result is positive for the presence of Listeria monocytogenes. This sixth step is performed when the Petri dish is positioned at the station PT6 and when it is “positive”.

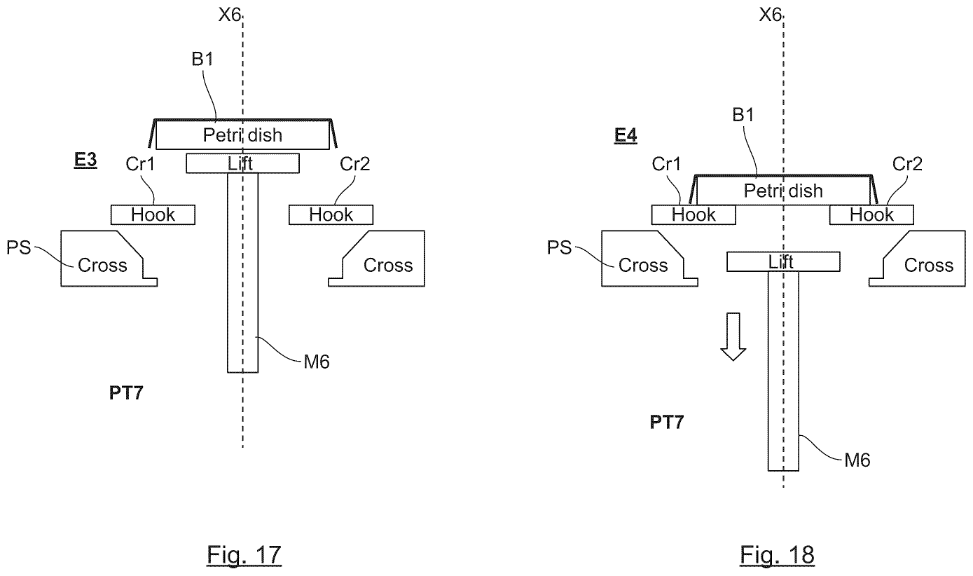

The station PT6 comprises a manipulator M6 mounted so as to move in translation along the transfer axis X6 between a position for picking up the base Fo from the lower plate PI (FIGS. 14-15), an intermediate position for securing the base Fo to the lid Co of the dish at the upper plate PS (FIG. 16) and a position for placing the assembled dish in the storage carousel CS2 (FIGS. 17-18). The manipulator M6 cooperates with a drive motor (not shown), which is controlled by the control unit and, when activated, facilitates the vertical movement thereof from a low position to a high position and vice versa from the high position to the low position. The manipulator M6 is dimensioned to be housed in the lower sliding surface SI, with its stroke passing through the openings of the lower plate PI and the upper plate PS.

On arrival at the station PT6, the base of the dish Fo is arranged on the lower plate PI while the lid Co is arranged on the upper plate PS (FIGS. 14 and 15). The manipulator M6 is then embedded in the lower sliding surface SI so as not to hinder the movement of the base Fo on the sliding surface SI. At the command of the control unit, the drive motor of the manipulator M6 is activated to facilitate its vertical movement and the ascent of the base Fo towards the storage column C6 of the carousel CS2. As it ascends, the manipulator M6 passes through the opening of the upper plate PS, so that the base Fo comes into contact with the lid Co remaining on the upper platform PS and joins with it with minimal friction. In order to ensure smooth reassembly of the base and lid, the speed of the manipulator is slowed down as it passes through the upper plate PS (FIG. 16). The manipulator M6 continues its ascent to pass through the opening in the lower plate of the carousel CS2, allowing the reassembled sorting dish to move through and then over a pair of anti-return hooks Cr1-Cr2, where it will be stacked with the other previously processed positive Petri dishes. These anti-return hooks are mounted on hinges to facilitate a passive pivot connection to the carousel plate, allowing them to move between a horizontal position that secures the dish in the storage column CS6 (FIG. 18) and an oblique (or nearly vertical) position that permits the dish to be transferred into the storage column CS6 (FIG. 16).

If the Petri dish is of a different type, it is not transferred by the station PT6. The manipulator M6 is not activated, and the base of the dish Fo and the associated lid Co remain in their respective housings.

The double plate is then activated by the control unit to move the Petri dish (base Fo and lid Co separated) by angular increments of 2π/8 about the axis X2, in order to transfer it to the station PT7.

Step 7: Transfer of “Positive” Dishes (FIG. 14)

This seventh step is performed when the Petri dish is positioned at the station PT7. This seventh step is identical to the sixth step and is carried out for a “positive” Petri dish in conjunction with the second storage carousel CS2, when the storage column C6 is full (i.e. forty dishes stacked here) and therefore cannot accommodate any other Petri dishes. In this specific embodiment, the station PT7 thus serves the same function as the station PT6 and operates as a duplicate when the storage column associated with the station PT6 is full, thereby increasing the storage capacity for positive dishes (i.e. eighty dishes in this case). The processing unit is configured to manage the counting of the positive Petri dishes transferred by the station PT6 into the column CL2 before activating the station PT7 for the transfer of other positive Petri dishes into the column CL3.

The station PT7 comprises a manipulator M7 that is identical to the manipulator M6, meaning it is mounted so as to move in translation along the transfer axis X7, cooperating with a drive motor (not shown), which is controlled by the control unit. The manipulator M7 is dimensioned to be housed in the lower sliding surface SI, with its stroke passing through the openings of the lower plate PI and the upper plate PS. At the command of the control unit, the activation of the drive motor initiates the vertical movement of the manipulator M7 and the ascent of the base Fo towards the storage column C6 of the carousel CS2, passing through the opening of the upper plate PS to secure it to its lid, and then through the opening of the lower plate of the carousel CS2, to position the reassembled sorting dish in the storage column C7 above a pair of anti-return hooks.

If the Petri dish is of a different type, it is not transferred by the station PT7. The manipulator M7 is not activated, and the base of the dish Fo and the associated lid Co remain in their respective housings.

The double plate is then activated by the control unit to move the Petri dish (base Fo and lid Co separated) by angular increments of 2π/8 about the axis X2, in order to transfer it to the station PT8.

Step 8: Transfer of “Negative” Dishes (figure 23)

This eighth step is performed when the Petri dish is positioned at the station PT8. This eighth step is the same as the sixth (or seventh) step and is carried out for a “negative” Petri dish in conjunction with the first storage carousel CS1.

The station PT8 comprises a manipulator M8 that is identical to the manipulator M6 (or M7), meaning it is mounted so as to move in translation along the transfer axis XR, cooperating with a drive motor (not shown), which is activated by the control unit when a “negative” Petri dish is present at this station. The manipulator M8 is dimensioned to be housed in the lower sliding surface SI, with its stroke passing through the openings of the lower plate PI and the upper plate PS. At the command of the control unit, the activation of the drive motor initiates the vertical movement of the manipulator M8 and the ascent of the base Fo towards the receiving column of the carousel CS1, passing through the opening of the upper plate PS to secure it to its lid, and then through the opening OR of the lower plate of the carousel CS1, to position the reassembled sorting dish in the storage column and to be stacked there with the other previously processed negative Petri dishes.

The double plate is then activated by the control unit to move the Petri dishes by angular increments of 2π/8 about the axis X2 as long as there are Petri dishes remaining to be processed.

Thus, the machine MT is designed so that, depending on the type assigned to the Petri dish, it is transferred to either the first or second storage carousel, enabling automatic sorting of the Petri dishes.

In practice, it is possible to carry out complete processing of a set of four hundred dishes autonomously, once the dishes are loaded into the storage carousel CS1, thanks to this clever cooperation between the mobile storage carousel CS1 and the drive device DER, which are activated synchronously for each stack of Petri dishes to be dispensed. The number of dishes is given here purely for illustrative purposes, and a greater or lesser number may certainly be considered, without departing from the scope of the invention, depending on the storage capacity of the carousel CS1. This storage capacity depends on the height and number of storage columns.

It should also be noted that the machine MT counts as many Petri dishes being processed as there are workstations within the operational unit, with all workstations functioning continuously except when the drive device is activated to rotate the Petri dishes relative to the various workstations.

In relation to FIG. 21, an alternative embodiment of the Petri dish drive device according to the invention is now set out. Unlike the drive device DER designed to hold Petri dishes in the upright position (FIG. 3), this drive device DER-2 is designed to hold Petri dishes placed upside down, meaning the lid is facing downwards and the base is facing upwards. This alternative embodiment is particularly advantageous, for example, when the analysis necessitates that the Petri dishes remain closed throughout the process to prevent any risk of contamination of the culture medium. For example, for mould analysis, it is essential that culture media are not contaminated by highly volatile spores that may be present on the surface of Petri dishes. Furthermore, as the hyphae cover all or part of the surface of the Petri dish and can overlay various mould colonies, it becomes very challenging to identify and distinguish between different types of mould. Observing the base of the Petri dish by turning it over allows for a clearer differentiation of the various types of mould, as their colonies are more distinctly visible beneath the hyphae when viewed from the underside of the Petri dish.

As for the device DER, the device DER-2 comprises a double drive plate composed of an upper drive plate PS-2 and a lower drive plate PI-2, substantially annular in shape, arranged in parallel and secured to each other by spacers. The device DER-2 also comprises an upper sliding surface SS-2 that partially extends beneath the plate PS-2 and a lower sliding surface SI-2 that extends beneath the plate PI-2. The sliding surfaces SS-2 and SI-2 are identical to those of the device DER-2. The drive plates PS-2 and PI-2 each feature a series of eight identical housings evenly spaced around the periphery of the device and aligned vertically in pairs.

Unlike the device DER, the series of eight upper housings of the device DER-2 designed to hold the Petri dishes do not feature a lid retaining rim (i.e. annular shoulder EA). Indeed, the eight housings of the plate PS-2 each comprise a through-hole having a first portion of conical shape (leading to a gradual cross-section reduction) and a second cylindrical portion dimensioned to allow the passage of the entire Petri dish (base and lid) through the housing. Thus, for a request to process Petri dishes upside down, it is sufficient to remove the double drive plate PS-2-PI-2 from the device DER-2 and replace it with the double drive plate PS-PI from the device DER, while the sliding surfaces remain identical from one embodiment to another.

FIG. 22 schematically shows in a simplified manner the structure of a Petri dish processing device in one specific embodiment, for example the processing unit that carries out the method according to the invention (for example, steps 1 to 8 described above in relation to FIGS. 5 to 20, 23).

This processing device 10 comprises a random-access memory 130 (for example, RAM), a CPU unit 110, equipped, for example, with a processor or microprocessor, and controlled by a computer program stored in a read-only memory 120 (for example, ROM or a hard disk). Upon initialisation, the code instructions of the computer program are loaded into the random-access memory 130, for example, before being executed by the processor of the CPU unit 110. Such a computer program allows the execution of at least one iteration of steps 1 to 8 described above (i.e. at least one processing cycle).

The processing unit 51 receives input commands and instructions for initiating the processing (indicated by arrow E) provided by the operator through the software interface. The processor of the CPI unit 51 then carries out the process based on the input commands E and outputs the corresponding control commands (indicated by the arrow S) to the various processing stations, in accordance with the instructions of the program 120, using the control unit of the machine.

This FIG. 22 shows only one specific method, among a plurality of possible options, for carrying out the various steps outlined above. Indeed, the processing method according to the invention can be carried out without preference:
on a reprogrammable computing machine (a PC, a DSP processor, or a microcontroller) running a program comprising a sequence of instructions; or
on a dedicated computing machine (for example, a set of logical gates such as an FPGA or an ASIC, or any other hardware module).

In the event that the invention is implemented on a reprogrammable computing machine, the corresponding program (i.e. the sequence of instructions) may be stored on a removable storage medium (such as a floppy disk, CD-ROM, or DVD-ROM) or otherwise, with this storage medium being partially or fully readable by a computer or processor.

1. An automated machine for processing culture dishes, wherein the machine comprises:
a first storage carousel mounted for rotation about a first axis, designed for the storage of culture dishes to be processed;
a second storage carousel mounted fixed about a second axis; and
an operational unit designed to receive culture dishes in continuous succession from the first carousel and to carry out a series of successive treatments on each of the received culture dishes, said operational unit comprising:
a plurality of stationary processing stations arranged at predetermined intervals around the second axis, with at least some of said processing stations being capable of being activated at least partially simultaneously, said processing stations comprising:
a receiving station intended to receive a culture dish to be processed from the first carousel;
an analysis and allocation station designed to analyse the contents of the received culture dish and allocate it the culture dish a specific type from at least two predefined dish types based on results of the analysis; and
at least one first and one second transfer station designed to transfer the culture dish to either the first or second storage carousel, depending on the type allocated to said culture dish; and
a drive device for rotating the culture dishes relative to the processing stations, said drive device comprising housings for the culture dishes arranged at intervals corresponding to those of the processing stations, such that a rotation of the drive device about the second axis successively positions a given culture dish at each of the processing stations.

2. The automated machine according to claim 1, wherein said drive device comprises an upper drive plate and a lower drive plate arranged parallel to one another and integral with one another, the upper and lower drive plates being mounted for rotation about the second axis and comprising a set of upper and lower Petri dish housings respectively, the upper and lower housings being evenly distributed on the a periphery of the upper and lower drive plates around the second axis, and arranged facing each other in pairs.

3. The automated machine according to claim 2, wherein said drive device further comprises:
an upper sliding surface fixedly mounted in relation to the second axis and extending partially along a lower face of the upper drive plate, and
a lower sliding surface fixedly mounted in relation to the second axis and extending along the lower face of the lower drive plate.

4. The automated machine according to claim 3, wherein the culture dishes to be processed are each provided with a lid attached to a base and positioned in an upright position in the first carousel and wherein:
the upper housings are each provided with a through-hole dimensioned to allow the passage of a culture dish and the lower end of which ends with an annular shoulder dimensioned to retain the lid in the upper housing and allow the base to separate from the lid due to gravity in absence of the upper sliding surface, the upper sliding surface extending partially under the upper drive plate to hold the base attached to the lid on at least one of the processing stations;
the lower housings are each provided with a through-hole dimensioned to allow passage of a culture dish base, the lower sliding surface extending under the lower drive plate to secure the base in the lower housing;

5. The automated machine according to claim 4, wherein the stationary processing stations further comprise a culture dish opening station comprising:
a flap mounted so as to move between at least one position for holding the base attached to the lid and a release position allowing the base to be separated from the lid;
manipulator mounted so as to move in translation between at least one position for picking up the base and a position for placing the base on the lower plate;
the flap comprising an opening for the introduction of the manipulator, said picking up position being assumed when the flap is in the base holding position after the manipulator has passed through the introduction opening of the flap.

6. The automated machine according to claim 5, wherein the flap is mounted so as to rotate about a vertical axis of an actuator which is configured to exert pressure on a pin arranged in an opening passing through the drive device, said pin exerting pressure on the lid when the flap is in the release position.

7. The automated machine according to claim 3, wherein said at least one first and second transfer stations each comprise a manipulating member that is mounted so as to move in translation between at least one position for picking up the base from the lower drive plate, an intermediate position for securing the base to the lid at the upper drive plate, and a position for placing the base and the lid, secured together, in either the first or second storage carousel.

8. The automated machine according to claim 3, wherein the stationary processing stations further comprise at least one culture dish identification station belonging to the group consisting of: a front bar code reader identification station and a side bar code reader identification station, said upper sliding surface covering the lower face of the upper drive plate on the receiving station and said at least one identification station.

9. The automated machine according to claim 1, said at least one first transfer station is arranged to transfer to the first carousel the culture dishes allocated a first type and said at least one second transfer station is arranged to transfer to the second carousel the culture dishes allocated a second type.

10. The automated machine according to claim 9, wherein the culture dish opening station is additionally designed to transfer to the first carousel the culture dishes allocated a third type related to a processing issue, the manipulator of said opening station being further mounted to move in translation between a position, among said picking up position and said placing position, and a dish transfer position in the second storage carousel.

11. The automated machine according to claim 3, wherein the culture dishes to be processed are each provided with a lid attached to a base and positioned in a so-called upside down position in the first carousel and wherein:
the upper housings are each provided with a through-hole dimensioned to allow passage of a culture dish, the upper sliding surface extending partially under the upper drive plate to hold the culture dish closed in the upside down position in the upper housing on at least one of the processing stations;
the lower housings are each provided with a through-hole dimensioned to allow the passage of a culture dish, the lower sliding surface extending under the lower drive plate to support the base and hold the culture dish closed in the upside down position in the lower housing.

12. A method comprising:
processing culture dishes using an automated processing machine comprising a first storage carousel, a second storage carousel, and an operational unit designed to receive the culture dishes in continuous succession and to carry out a series of successive treatments on each of the culture dishes, the operational unit comprising a plurality of stationary processing stations arranged at constant intervals around an axis of rotation and a drive device for rotating the culture dishes relative to the processing stations, said drive device comprising housings for the culture dishes arranged at intervals corresponding to those of the processing stations, such that a rotation of the drive device about the second axis successively positions a given culture dish at each of the processing stations in order to carrying out the following steps, at least partially simultaneously:
receiving a culture dish to be processed from the first carousel;
analysing contents of the received culture dish and allocating to said dish a specific type from at least two predefined types of dish based on results of the analysis; and
transferring the culture dish, the contents of which have been analysed, to either the first or second storage carousel, depending on the type allocated to said culture dish.

13. (canceled)

14. A non-transitory computer readable storage medium, storing a computer program product comprising program code instructions for implementing the method according to claim 12, when the program is executed on at least one processor of the automated processing machine.
